(19) United States
(12) Patent Application Publication
(10) Pub. No.: US 2025/0134294 A1
(43) Pub. Date: May 1, 2025

(54) SYSTEM AND METHOD FOR INTEGRATED PRAYER RUG

(71) Applicant: Kirstine Stewart, Toronto (CA)

(72) Inventor: Kirstine Stewart, Toronto (CA)

(21) Appl. No.: 18/915,718

(22) Filed: Oct. 15, 2024

Related U.S. Application Data
(60) Provisional application No. 63/590,812, filed on Oct. 17, 2023.

(51) Int. Cl. A47G 33/00; A47G 27/02; G16H 20/30; G16H 20/70; H04W 4/80

(52) U.S. Cl. CPC A47G 33/008; A47G 27/0237; G16H 20/30; G16H 20/70; H04W 4/80

(57) ABSTRACT

The present disclosure relates to an integrated prayer rug system, comprising a rug comprising an NFC chip (or other RFID devices, Bluetooth, proximity sensors, or electronic thread technology) in communication with an interface device having a computer processor and memory storing computer code wherein the interface device has internet communication capability and is chosen from a smartphone, a computer, a tablet computer, or a smartwatch; and an app to capture information before, during or after each use of the system and which is utilized by the app to integrate with the user's overall health, exercise, and spiritual wellbeing.

### CROSS-REFERENCE TO RELATED APPLICATION

This application claims priority to U.S. Provisional Appl. Ser. No. 63/590,812 filed on Oct. 17, 2023, which is incorporated herein by reference in its entirety to the full extent permitted by law.

### TECHNICAL FIELD

The present invention relates to the field of electronically connected prayer rugs to integrate faith, meditation and prayer in a more accessible technology than heretofore known.

### BACKGROUND

Digital technology has brought a wealth of benefits to our work and home lives and accelerated the pace of life dramatically for billions of people around the world. Artificial intelligence (AI), Machine Learning, Metaverse, Web3, and immersive technology are merging the physical and virtual worlds. We wear sport trackers, we live in smart houses, and we connect to all kinds of devices, apps and technologies that optimize the elements of work and home. The pandemic shifted the use of these technologies. Confined to home, attention focused on the activities that four square walls could accommodate. Shopping, deliveries, health care, education and work all transitioned to a web of connected apps, videos and chats.

Post-pandemic, things are different again. A successful re-entry into life “outside” relies on finding balance in a world where the physical and digital are more intertwined, and the pace is faster than ever before. The aftereffects of the pandemic show a marked increase in general physical and mental health awareness. An Ipsos poll showed that 62% of Americans surveyed believe that health is more important to them than it was prior to the pandemic. In response, the wellness market has exploded. People searching for solutions to physical and mental wellness challenges pushed the wellness global market size to $1.5 Trillion with annual growth of up to 10% (Mckinsey Insights Wellness report). A significant 92% of employers have expanded support for mental wellness for employees in 2021 (HR Executive Jun. 10, 2021). However, it is increasingly difficult for health-conscious consumers to find what is truly effective. The overwhelming scope of wellness offerings can be complex and confusing. People crave simplicity to help them cope with busy home, family and work lives. Spirituality is a significant part of people's lives and the positive impact of religion on health is profound, finding that it can improve overall mental health. There is, therefore, is a need in the art for a meaningful lifestyle solution that integrates our physical and spiritual lives with our digital.

### SUMMARY

The present disclosure satisfies the above need and relates to systems and methods for integrating faith, meditation and prayer in a more accessible technology than heretofore known. The systems and methods of the present disclosure are designed to create a habit of elevated spiritual practice, and integrate with the data points we already track, like our steps, our heartbeat, and our exercise time. In this way, the present invention builds a complete map of total wellness—“body and soul”—from the physical to the spiritual by providing the first-ever digitally integrated prayer and meditation rug that supports, guides and helps keep track of one's spiritual practice.

In one embodiment, the system comprises a prayer rug having integrated electronics to connect to other electronic mobile devices like a smartphone, tablet or watch. The rug has electronic connectivity to a mobile application or app that unlike a fitness tracker is a “soul” tracker. The app, in one embodiment, is designed to motivate the user, share information, assesses activity on the rug in order to score activity, and connects to other apps and integrates data used for work and home like Slack, Apple Health, etc., to create a full “body and soul” map of personal health and well-being.

In another embodiment, the app, connected to the rug, engages with a broader connected community of users with immersive and real life experiences, e.g., among fellow practitioners with thought leaders, influencers/celebrities, artists, social and cultural experts.

The practice of “dua” (prayer, meditation, mantra) primarily happens during the daily prayers which take place on a prayer rug. In one embodiment, the rug of the present disclosure is sized about 2 feet×3 feet, although the size can vary. The rug is significant in Muslim prayer and relates to elements like time, cleanliness (wudu), geographical positioning and design and cultural and regional representation. The present disclosure comprises a digitally integrated prayer and meditation rug that supports, guides, and helps keep track of one's spiritual practice. The rug is lightweight, and resistant to microorganisms (e.g., employing manufactured microban technology).

In one embodiment, the app seamlessly connects the rug to other devices via digital handshake, i.e., an exchange of signals between devices ensuring synchronization whenever a connection, as with another device, is initially established. Such connection can be with one's calendar, smartwatch, smartphone and other devices/websites to provide a picture of one's whole health—body, mind and spirit. The present disclosure describes where tradition meets technology, transforming a user's meditative mat and prayer rug into a virtual “magic carpet” where one finds moments to reflect, to refocus and surround oneself in beautiful immersive experiences.

In one embodiment, the app pairs the unparalleled beauty of an artisan-crafted rug to the tech advances that will support and give one's practice an elevated level of intention. It may use machine learning (ML) to assess one's connected calendar, and suggest and schedule practice times. Generative AI may provide a novel experienced level of guidance and support to one's personal preferences and practice, whether he/she is at home or on the road. The system of the present disclosure provides feedback when integrated with one's other trackers to give one the entire picture of their health and well-being and contextualizing the rewards and benefits of their practice.

In another embodiment, the system leverages ML by assessing one's connected calendar to suggest and schedule practice times, and delivering real-time feedback so one can see the benefits of his/her practice. Connecting the rug by NFC chip (or other means such as Bluetooth) to one's mobile devices (Android, IOS, smartphone, tablet, and laptop) it links with the app, which then supports and guides one's practice. The app will check times in one's connected calendar to schedule and prompt your actions and keep track of one's practice achievements, providing motivation to help one meet his/her goals.

The app and system are capable of providing helpful feedback by integrating with the other trackers (e.g., Fitbit, Apple Health Kit, Oura, etc.) and builds a comprehensive picture of one's health beyond just the physical. It shows the practice benefits to your body, mind and spirit.

Additional embodiments of the present methods, compositions and the like will be apparent from the following description, drawings, examples, and claims. As can be appreciated from the foregoing and following description, each and every feature described herein, and each and every combination of two or more of such features, is included within the scope of the present disclosure provided that the features included in such a combination are not mutually inconsistent. The order of method steps stated herein are non-limiting and may vary yet achieve the objectives of the present invention. In addition, any feature or combination of features may be specifically excluded from any embodiment or aspect. Additional aspects and embodiments are set forth in the following description and claims, particularly when considered in conjunction with the accompanying examples and drawings.

### BRIEF DESCRIPTION OF THE FIGURES

FIG. 1 is an illustration of a prayer/meditation rug connected to a smartphone and a smartwatch according to one embodiment of the present disclosure.

FIG. 2 is an illustration of a prayer/meditation rug connected to a smartphone and a smartwatch according to one embodiment of the present disclosure.

FIG. 3 is an illustration of a prayer/meditation rug connected to a smartphone and a smartwatch according to one embodiment of the present disclosure.

FIG. 4 is an illustration of a prayer/meditation rug connected to a smartphone and a smartwatch according to one embodiment of the present disclosure.

FIG. 5 is an illustration of a screenshot of the app's metrics according to one embodiment of the present disclosure.

FIG. 6 is an illustration of a screenshot from the app according to one embodiment of the present disclosure.

FIG. 7 is a flowchart of the information architecture according to one embodiment of the present disclosure.

FIG. 8 is a screenshot of the app (DuaWalla™) on an iPhone according to one embodiment of the present disclosure.

FIG. 9 is an illustration of a screenshot from the app according to one embodiment of the present disclosure.

FIG. 10 is an illustration of a screenshot from the app according to one embodiment of the present disclosure.

FIG. 11 is an illustration of a screenshot from the app according to one embodiment of the present disclosure.

FIG. 12 is an illustration of a screenshot from the app according to one embodiment of the present disclosure.

FIG. 13 is an illustration of a screenshot from the app according to one embodiment of the present disclosure.

FIG. 14 is an illustration of a screenshot from the app according to one embodiment of the present disclosure.

FIG. 15 is an illustration of a screenshot from the app according to one embodiment of the present disclosure.

FIG. 16 is an illustration of a screenshot from the app according to one embodiment of the present disclosure.

FIG. 17 is an illustration of a screenshot from the app according to one embodiment of the present disclosure.

FIG. 18 is an illustration of a screenshot from the app according to one embodiment of the present disclosure.

FIG. 19 is an illustration of a screenshot from the app according to one embodiment of the present disclosure.

FIG. 20 is an illustration of a screenshot from the app according to one embodiment of the present disclosure.

FIG. 21 is an illustration of a screenshot from the app according to one embodiment of the present disclosure.

FIG. 22 is an illustration of a screenshot from the app according to one embodiment of the present disclosure.

FIG. 22 is an illustration of a screenshot from the app according to one embodiment of the present disclosure.

FIG. 23 is an illustration of a screenshot from the app according to one embodiment of the present disclosure.

FIG. 24 is an illustration of a screenshot from the app according to one embodiment of the present disclosure.

FIG. 25 is an illustration of a screenshot from the app according to one embodiment of the present disclosure.

FIG. 26 is an illustration of a screenshot from the app according to one embodiment of the present disclosure.

FIG. 27 is an illustration of a screenshot from the app according to one embodiment of the present disclosure.

FIG. 28 is an illustration of a screenshot from the app according to one embodiment of the present disclosure.

FIG. 29 is an illustration of a screenshot from the app according to one embodiment of the present disclosure.

FIG. 30 is an illustration of a screenshot from the app according to one embodiment of the present disclosure.

FIG. 31 is an illustration of a screenshot from the app according to one embodiment of the present disclosure.

FIG. 32 is an illustration of a screenshot from the app according to one embodiment of the present disclosure.

FIG. 33 is an illustration of a screenshot from the app according to one embodiment of the present disclosure.

FIG. 34 is an illustration of a screenshot from the app according to one embodiment of the present disclosure.

FIG. 35 is an illustration of a screenshot from the app according to one embodiment of the present disclosure.

FIG. 36 is an illustration of a screenshot from the app according to one embodiment of the present disclosure.

FIG. 37 is an illustration of a screenshot from the app according to one embodiment of the present disclosure.

FIG. 38 is an illustration of a screenshot from the app according to one embodiment of the present disclosure.

FIG. 39 is an illustration of a screenshot from the app according to one embodiment of the present disclosure.

FIG. 40 is an illustration of a screenshot from the app according to one embodiment of the present disclosure.

FIG. 41 is an illustration of a screenshot from the app according to one embodiment of the present disclosure.

FIG. 42 is an illustration of a screenshot from the app according to one embodiment of the present disclosure.

FIG. 43 is an illustration of a screenshot from the app according to one embodiment of the present disclosure.

FIG. 44 is an illustration of a screenshot from the app according to one embodiment of the present disclosure.

FIG. 45 is an illustration of a screenshot from the app according to one embodiment of the present disclosure.

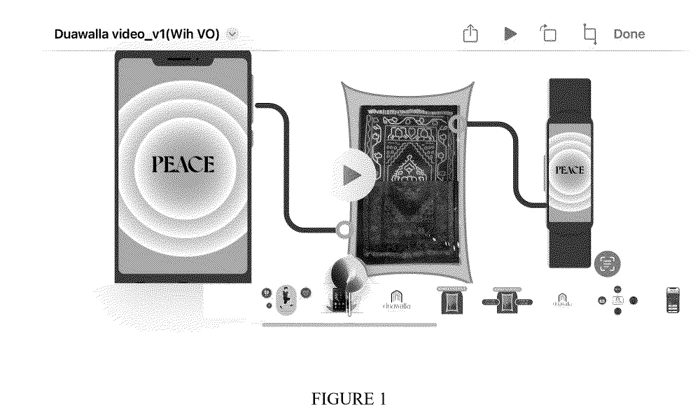

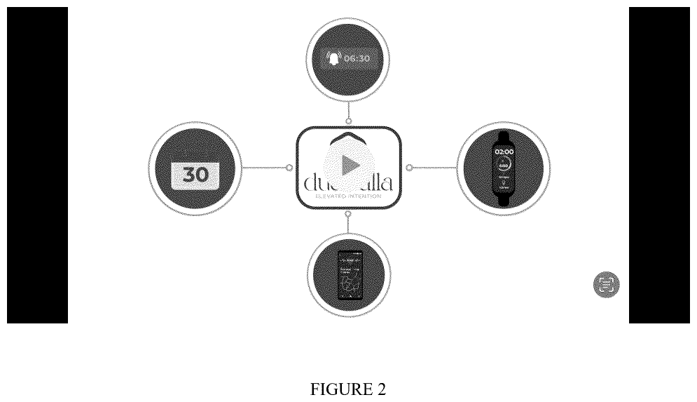

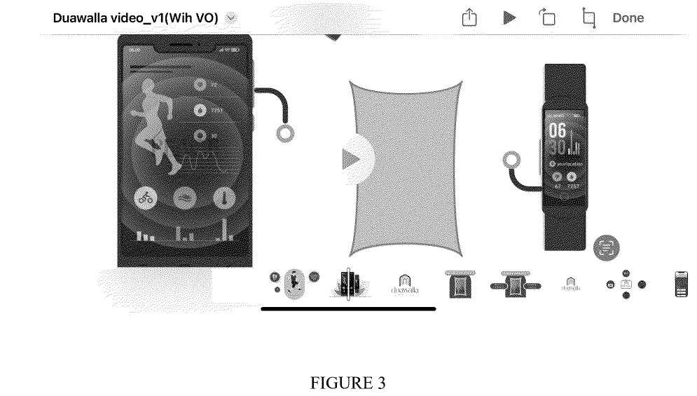

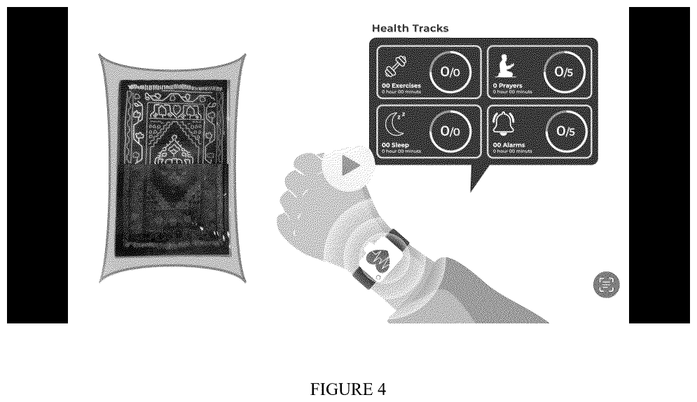

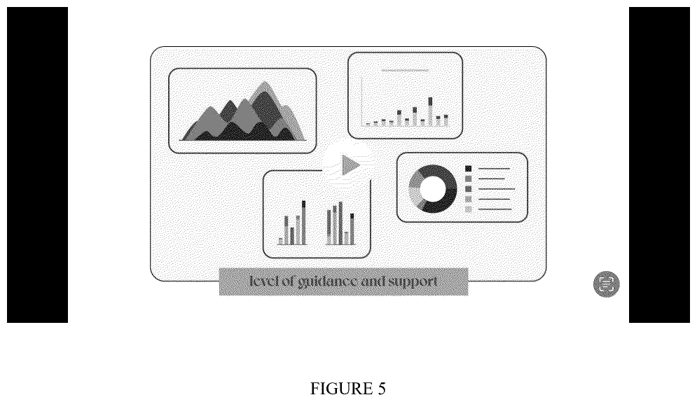

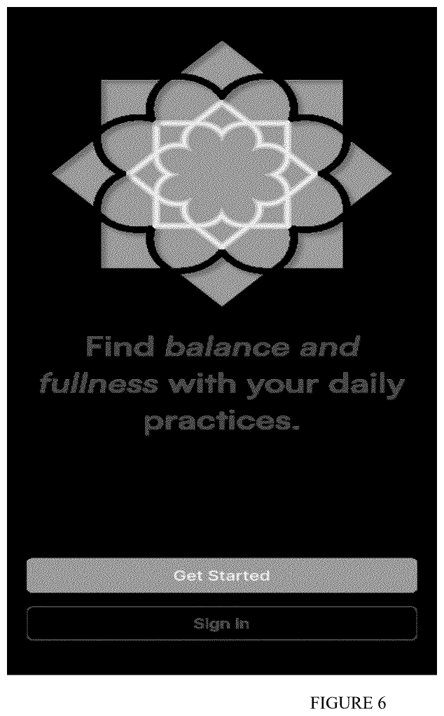

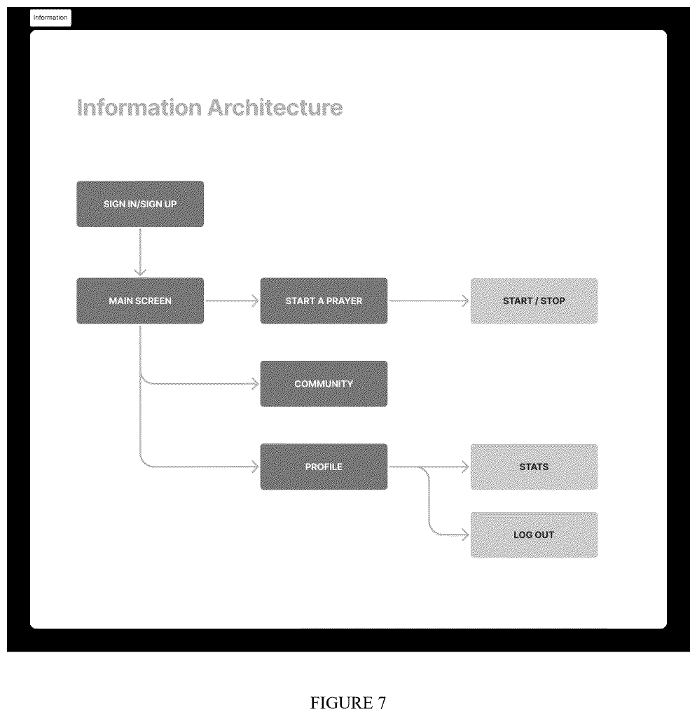

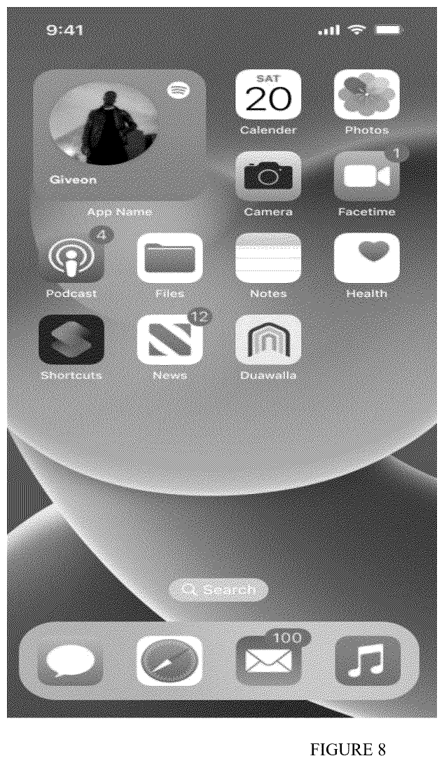

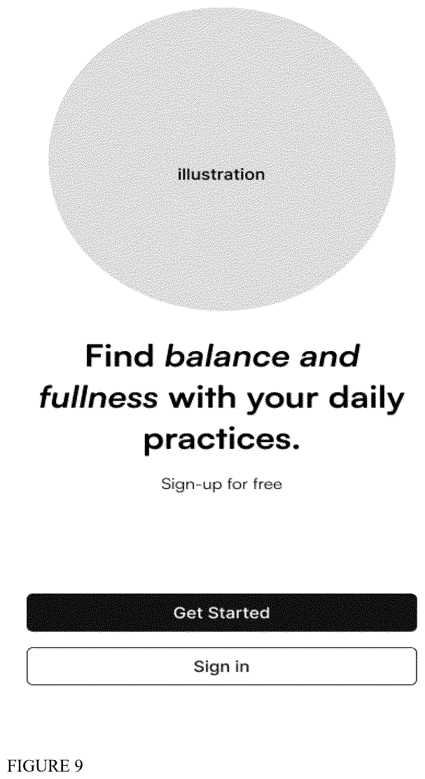

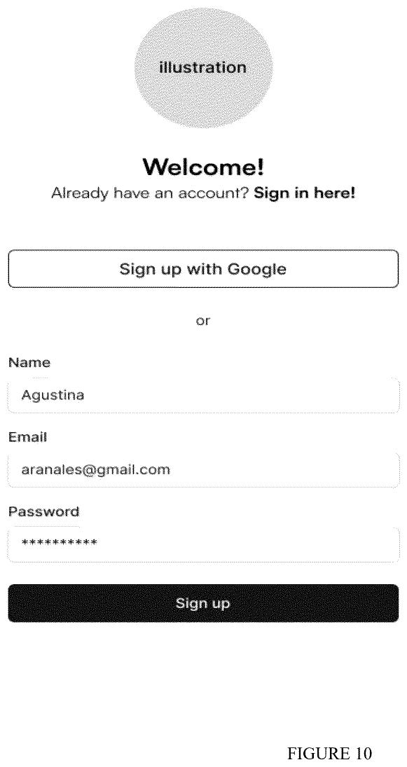

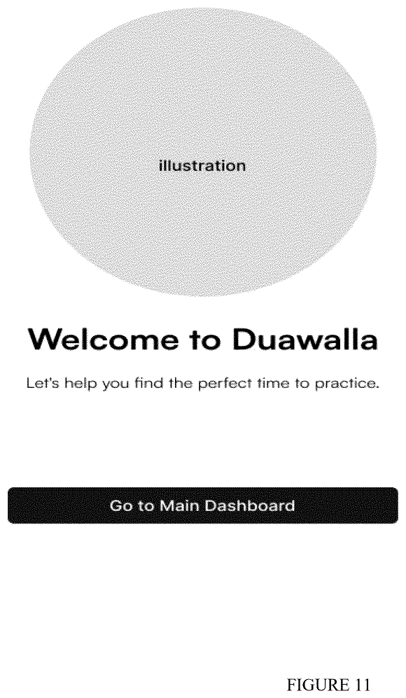

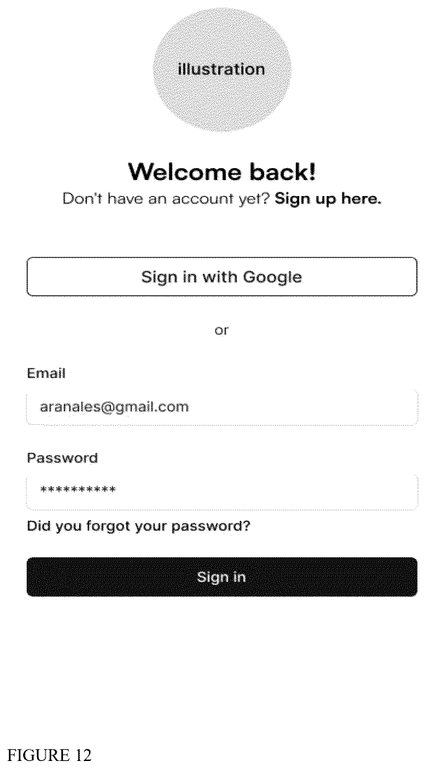

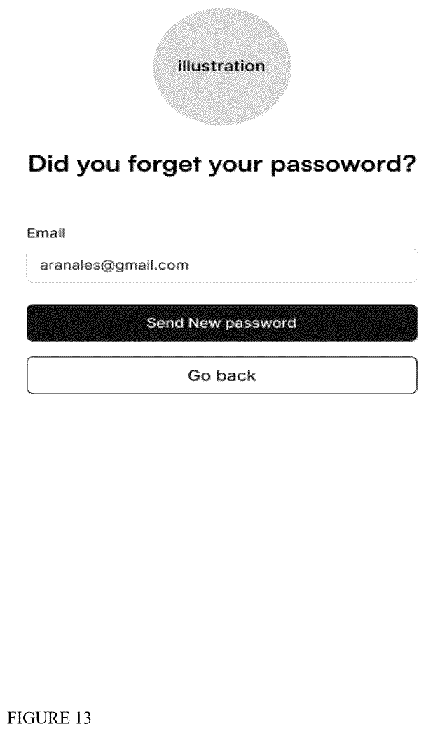

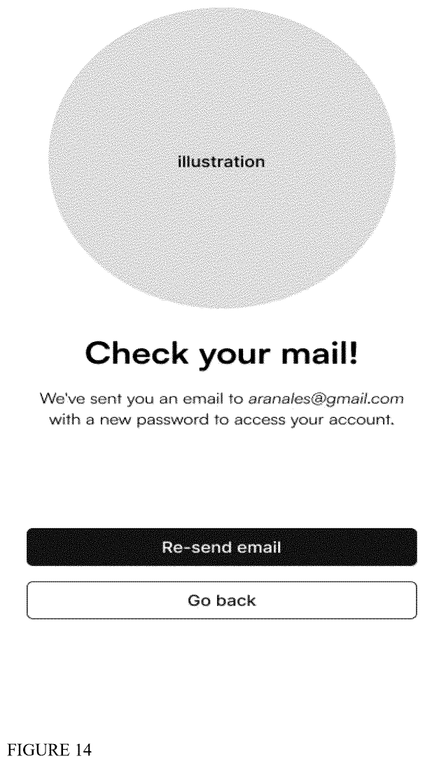

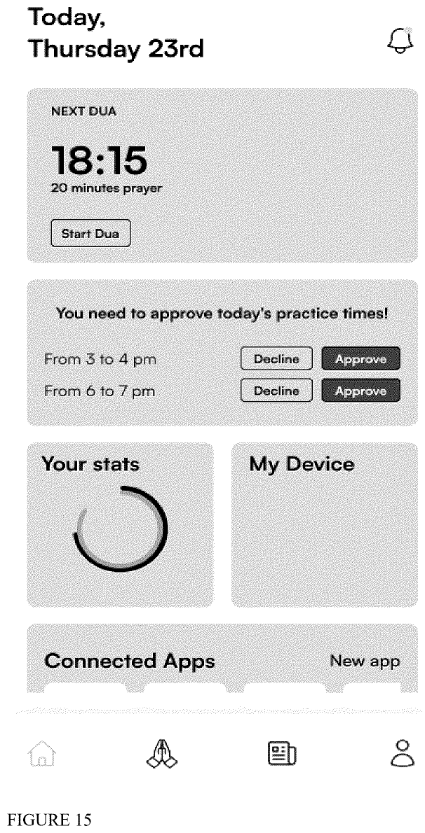

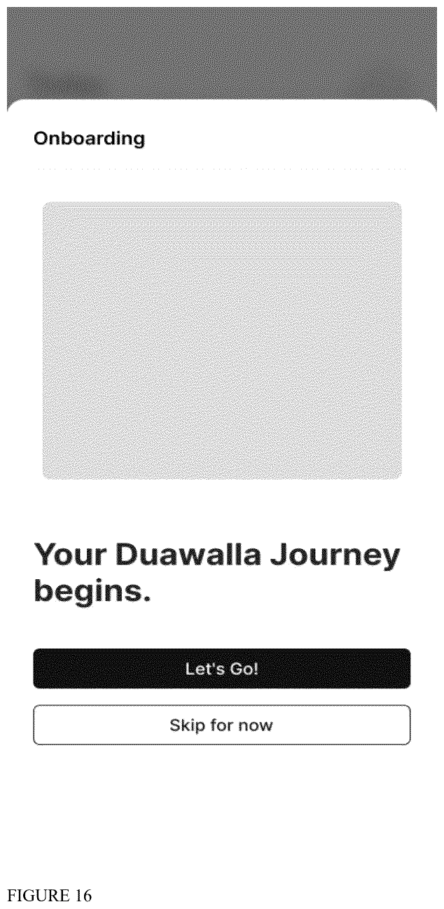

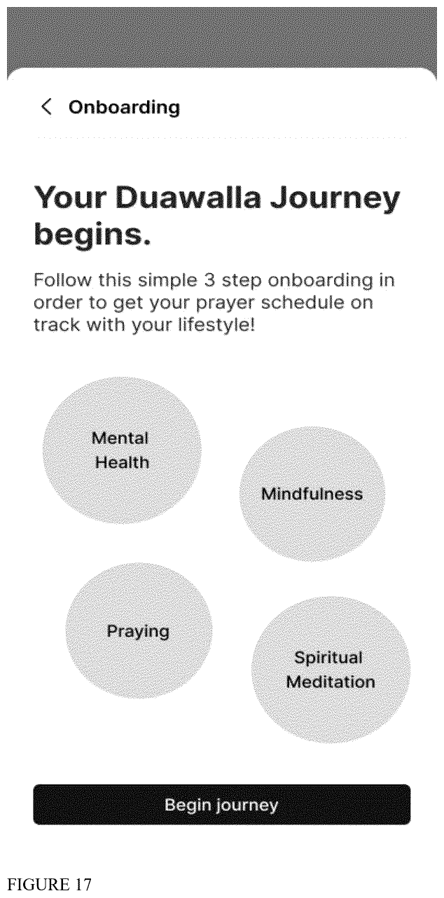

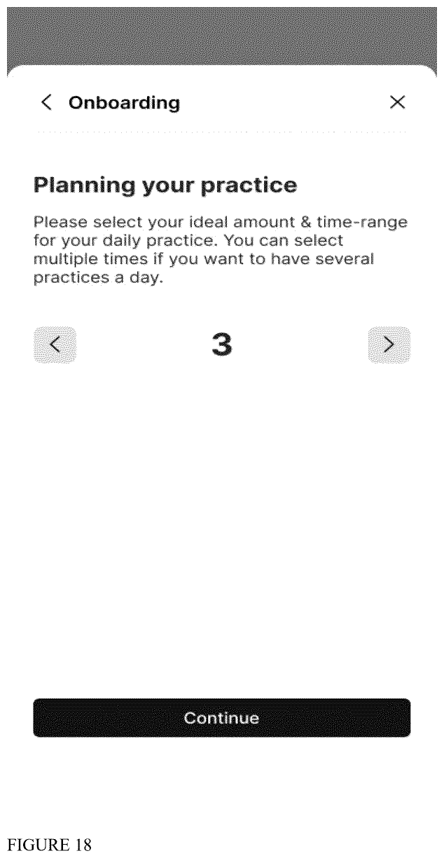

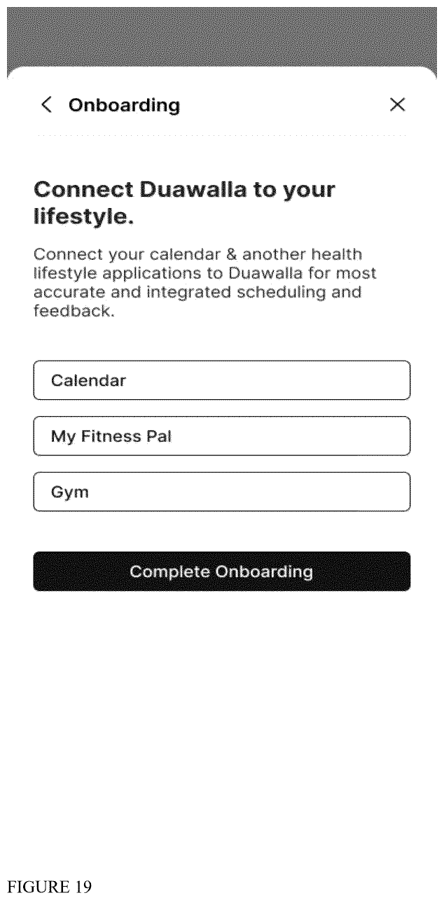

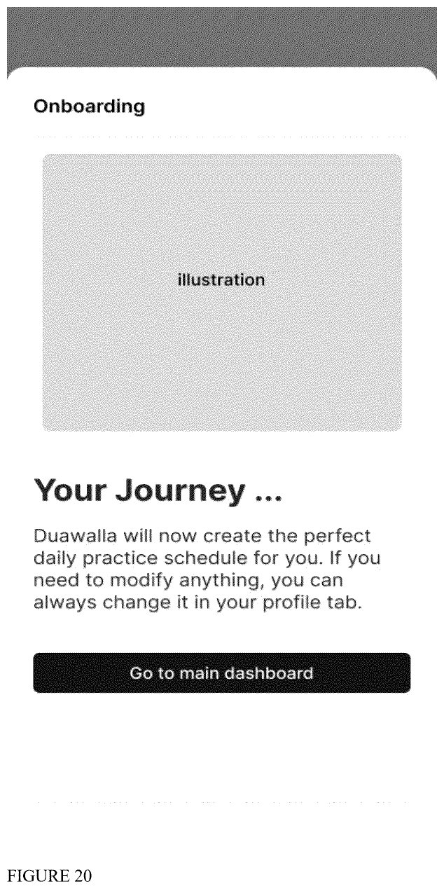

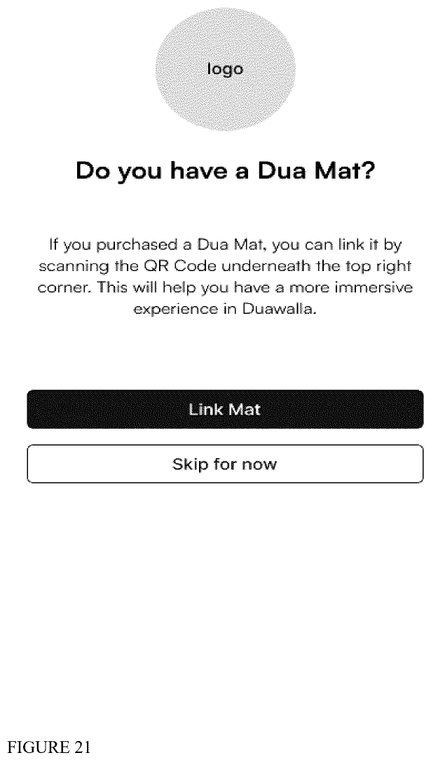

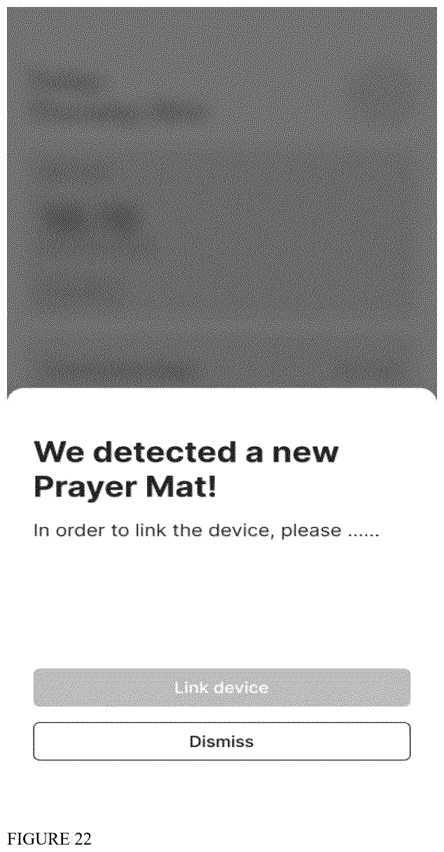

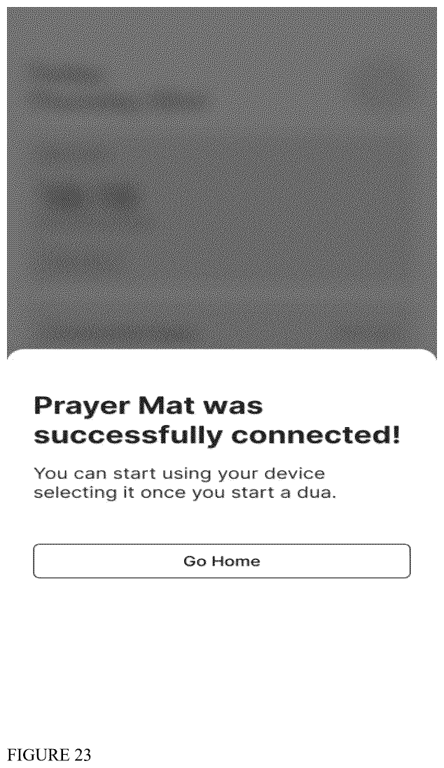

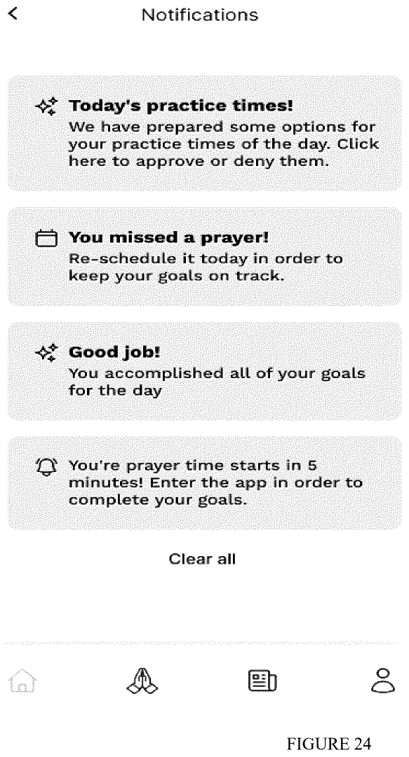

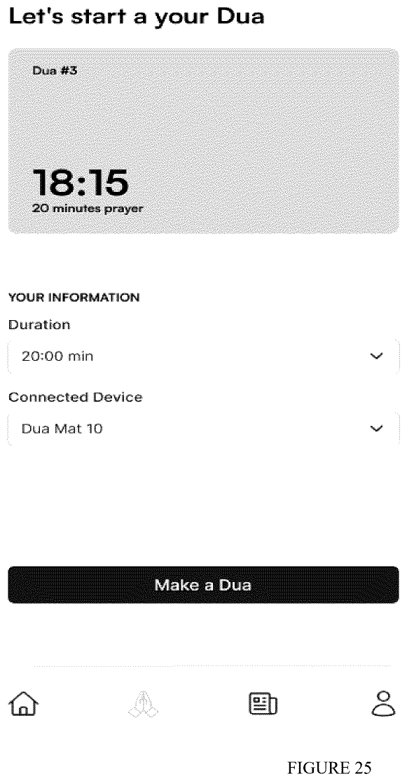

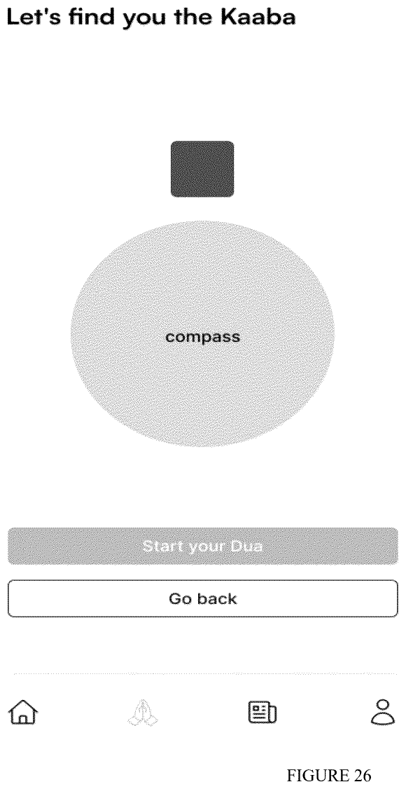

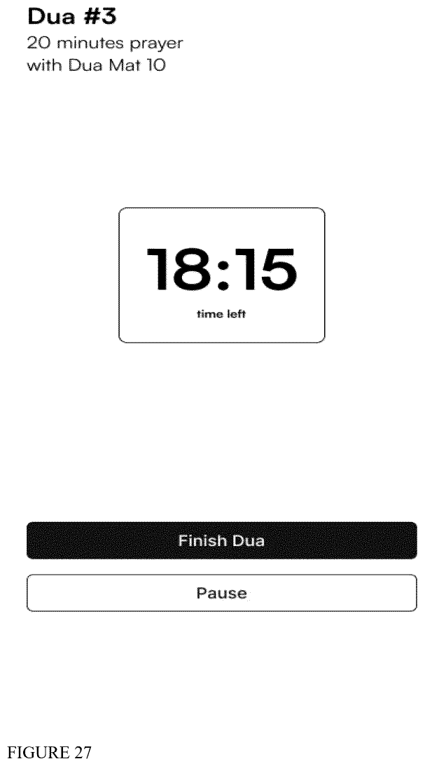

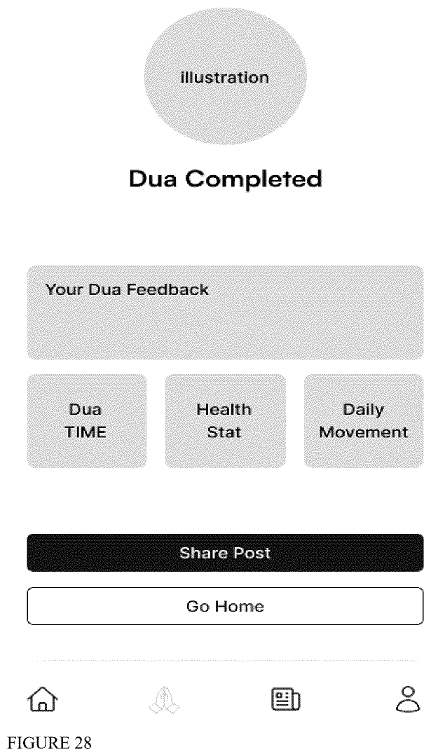

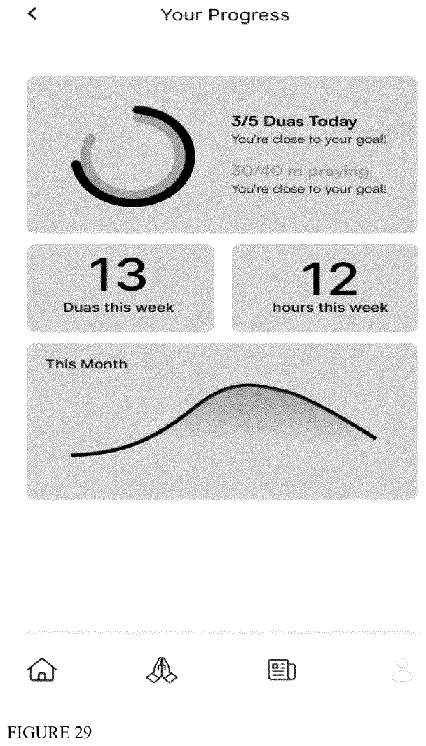

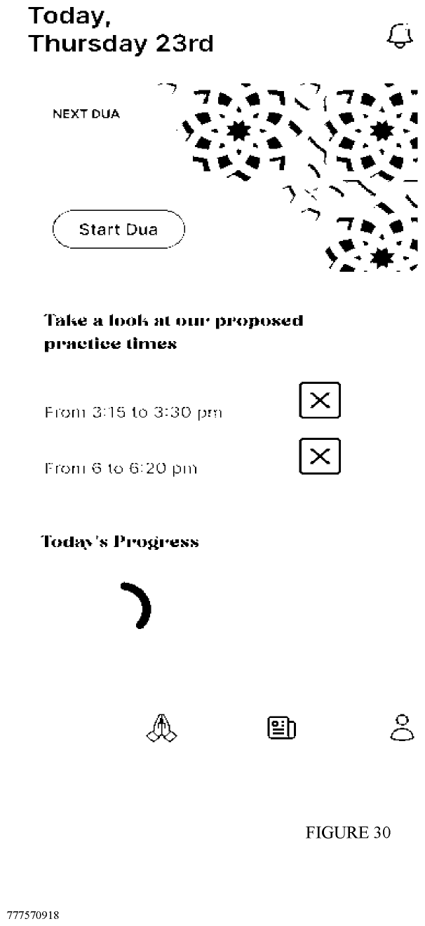

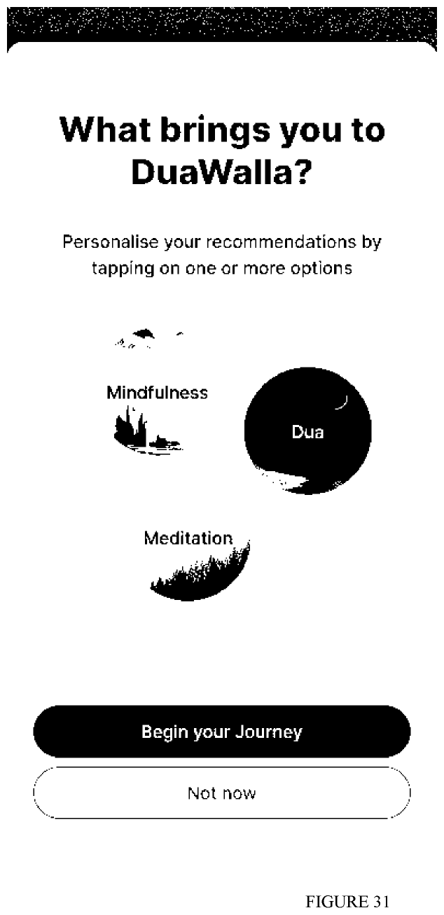

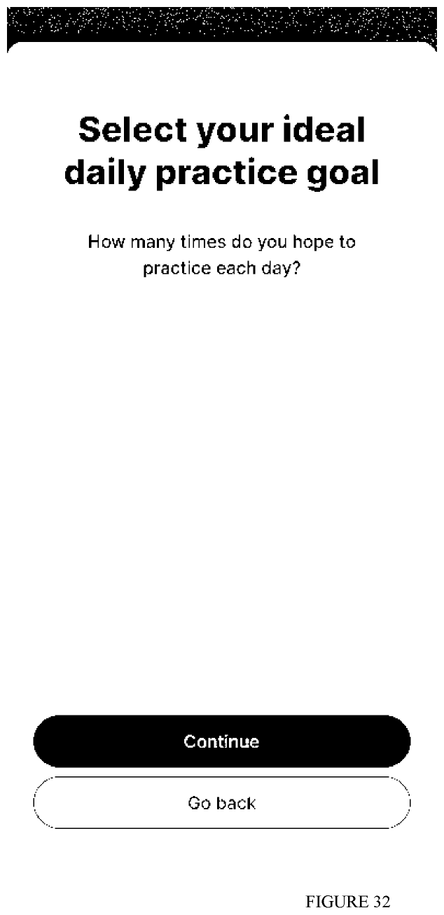

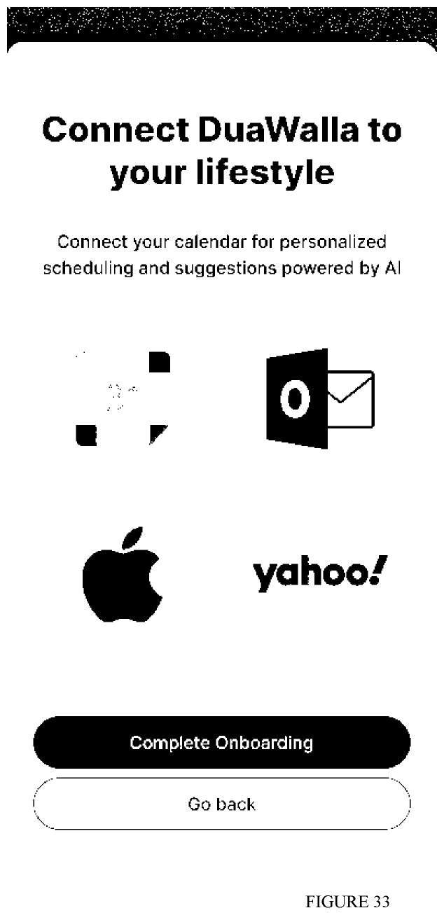

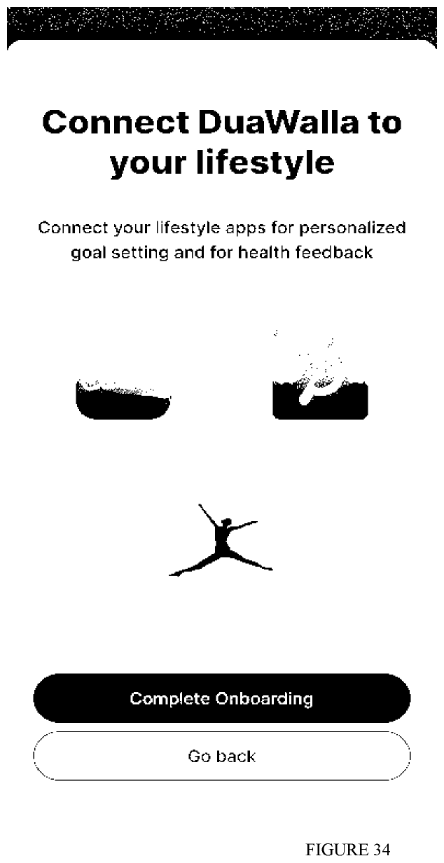

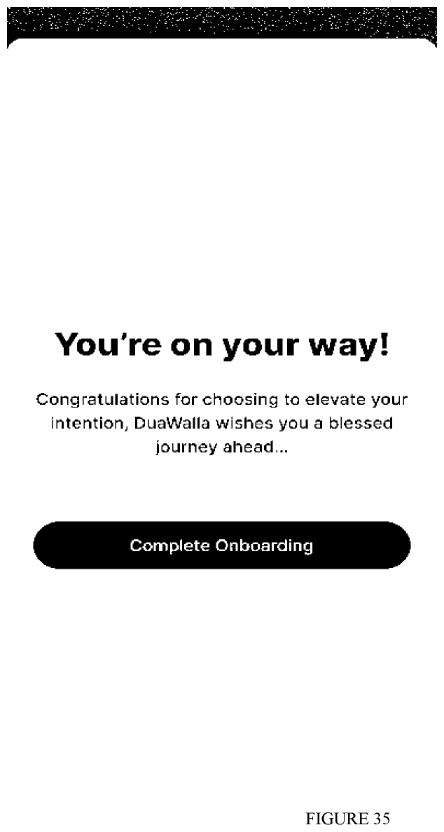

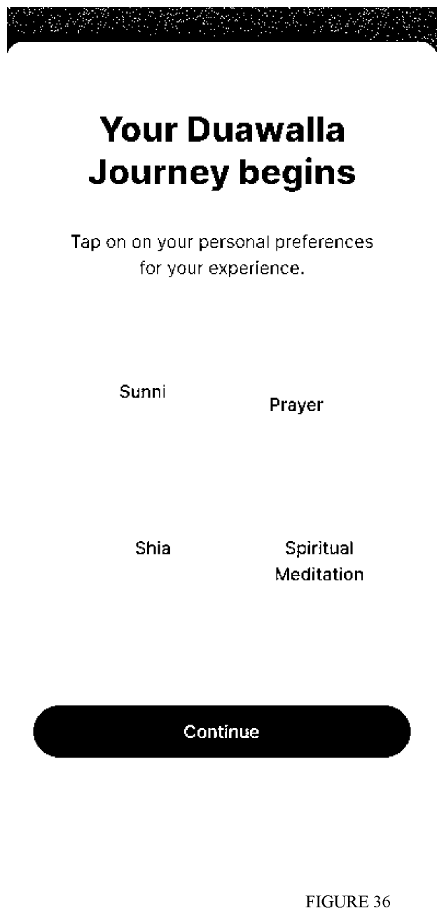

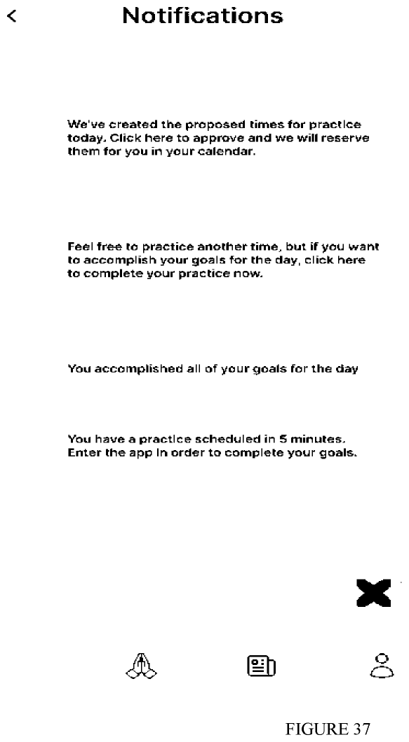

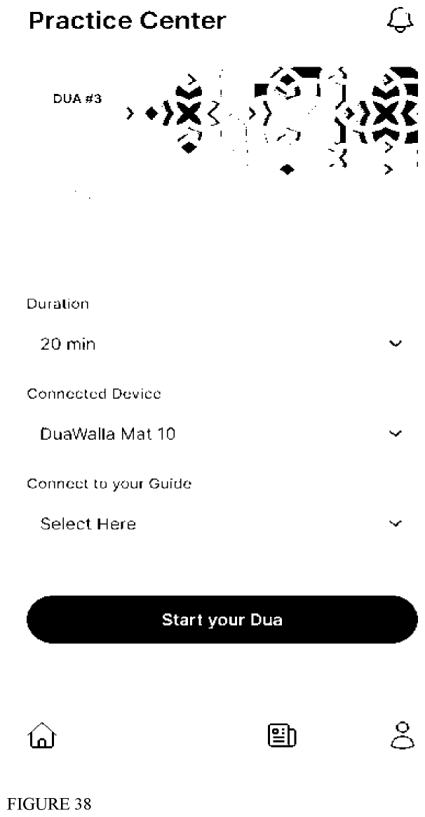

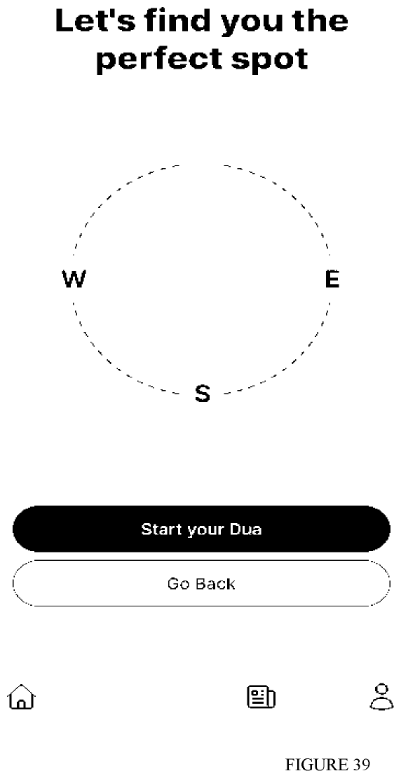

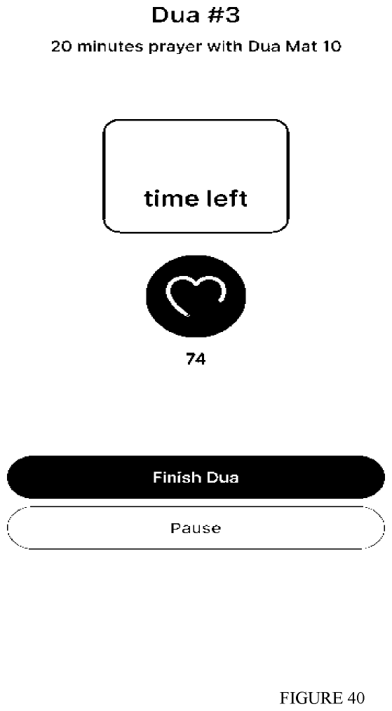

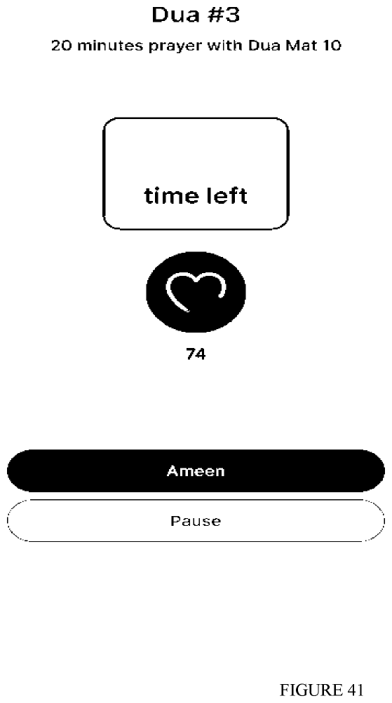

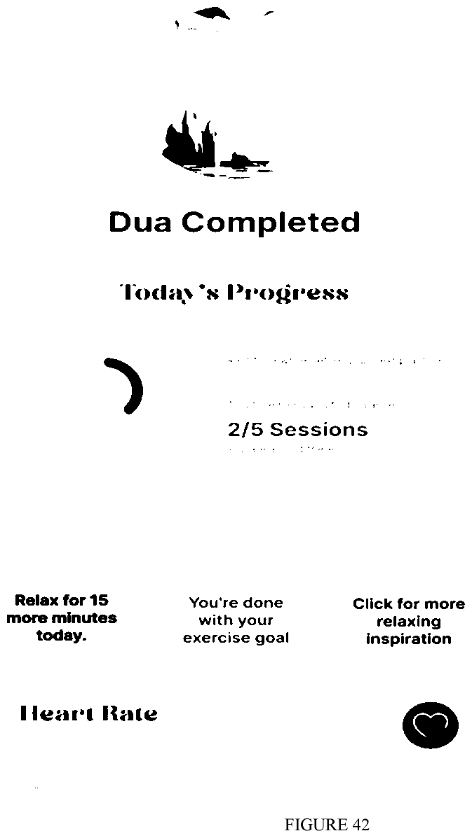

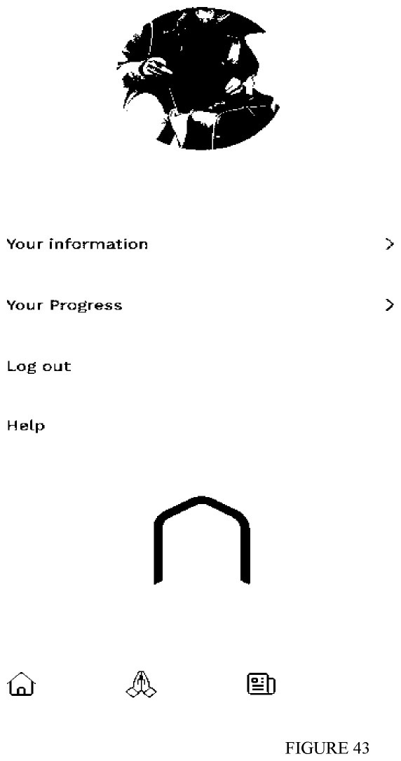

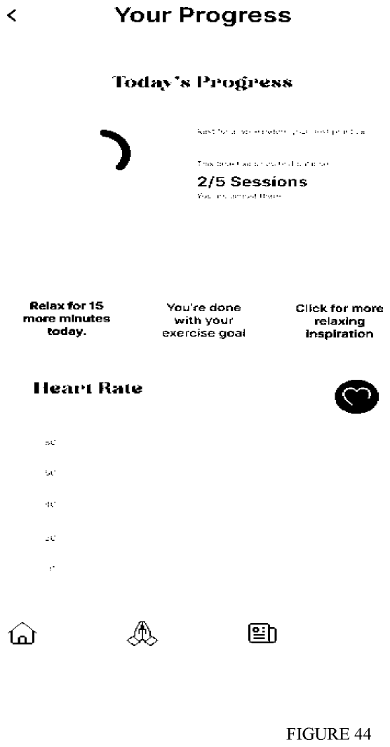

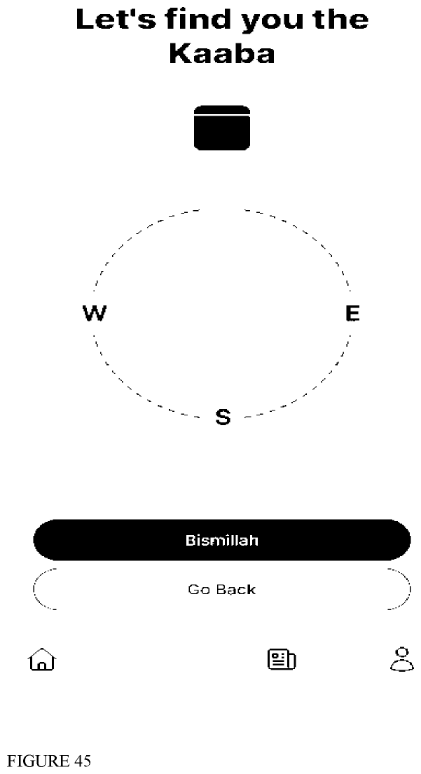

### DETAILED DESCRIPTION

The various aspects and embodiments will now be fully described herein. These aspects and embodiments may, however, be embodied in many different forms and should not be construed as limiting; rather, these embodiments are provided so the disclosure will be thorough and complete, and will fully convey the scope of the present subject matter to those skilled in the art. All publications, patents and patent applications cited herein, whether supra or infra, are hereby incorporated by reference in their entirety.

Unless defined otherwise, all terms and phrases used herein include the meanings that the terms and phrases have attained in the art, unless the contrary is clearly indicated or clearly apparent from the context in which the term or phrase is used. Although any methods and materials similar or equivalent to those described herein can be used in the practice or testing of the present invention, particular methods and materials are now described.

The systems, products and methods of the present disclosure address the modern-day needs of the population who make up the Muslim community, which is the fastest growing religion on the globe (and one of the most underserved markets by global and western marketers).

The prayer rug of the present disclosure may be of any size or shape suitable for meditation and prayer, e.g., 2 feet×3 feet; 2.5×4 feet; 3 feet×5 feet. The rug is portable, made of high quality, sustainably-made materials and has microbacterial and microorganism protection. In one embodiment, the design of the rugs are by global artisans to create beautiful, artistic and innovative designs to bring new expression to the rug and prayer experience.

The rugs comprise connectivity via a Near-Field Communication chip (“NFC chip” or “NFC chipset”), which is a silicon component or Integrated Circuit (IC) that can be used in different ways, depending on the targeted application. When connected to an appropriate antenna, an NFC chip enables short-range, wireless communication between two or more devices. In this case, the devices may be the prayer rug and a user's smartphone and/or smartwatch.

In one embodiment, the NFC chip is located near the top of the rug and senses the user's use of the rug, although the NFC chip may be placed at any location in the rug sufficient to collect and send the necessary information to connected devices, such as fitness trackers, smartphones, watches, tablets, work productivity apps, social media, etc. Other means to connect the rug with the devices may be employed, such as other RFID devices, Bluetooth, proximity sensors, and electronic thread technology.

In one embodiment, the app of the present disclosure will “wake up” on approach to the rug and connects to its tracking technology. It will also connect on the back end to other commonly used apps and devices (fitness trackers, smartphones, watches, tablets, work productivity apps, social media, etc.), helping to manage schedules and merge data points into a complete “body and soul” wellness map.

The app may use AI to deliver personalized content, tailoring the individual practice experience. Such content may be from prompts to ML sourced instruction, translation, pronunciation, context, scholarship and even automated discussion. Incorporating emerging AR/VR technology (like Apple Vision Pro) unlocks a truly immersive experience. For Muslims unable to break their business day to attend mosque, or who want to experience prayer in notable places like the Blue Mosque in Istanbul, they would travel on their carpet via immersive technology.

In one embodiment, the present disclosure provides for an integrated prayer rug system, comprising: (a) a rug comprising an NFC chip (or other RFID devices, Bluetooth, proximity sensors, or electronic thread technology) in communication with an interface device having a computer processor and memory storing computer code wherein the interface device has internet communication capability and is chosen from a smartphone, a computer, a tablet computer, or a smartwatch; and (b) an app to execute stored computer code on the interface device.

The app may be designed or uniquely tailored to a specific user and comprises: (i) a biometric authentication module to authenticate the user and to ensure digitally captured data is from the user; (ii) a prayer and/or meditation module having a prayer and/or meditation plan and content for the user; (iii) a user reminder module; (iv) an application program interface (API) between the rug and the smartphone, the computer, the tablet computer, or the smartphone to link the rug to the app; (v) a user self-assessment screen(s) module; (vi) an interface device database module, comprising digitally-captured or user-entered prayer and/or meditation information, and information of the user's use of the rug and system; and (vii) a communications and reporting module to report user information on the smartphone, the computer, the tablet computer, or the smartphone, and to social media if the app is prompted to do so.

The system may utilize information comprising prayer information, meditation information, the nature and extent of the user's use of the rug, user self-assessment information, digitally captured physiological, psychological, health and lifestyle information, information of prayers used by the user, or environmental data information. The system captures such information before, during or after each use of the system and which is utilized by the app to integrate with the user's overall health, exercise, and spiritual wellbeing.

Further, the information analyzed by the app are chosen from when the rug is used and for how long, goals, respiratory rate, blood oxygen saturation, pulse, heart rhythm, blood pressure, gait, muscle spasms, skin temperature, and weight. In another embodiment, the data is captured by rug and/or the interface device and then prompts the user self-assessment module to ask the user one or more questions about his/her experience or condition. The app may store the user's goals and/or schedule for prayer and/or meditation.

In one embodiment, the app reminds the user to pray, meditate, and/or breathe. And, the app is designed to automatically connect or handshake with the user's other digital health, exercise, lifestyle monitoring and/or tracking devices. Additionally, the app may present user specific self-assessment and/or data input screen(s).

In a further embodiment, the app maintains a database of user responses captured by self-assessment and/or data input screen(s) and digitally captured values and, optionally, scores the user's activity on the rug.

The app may further comprise a GPS module to record the user's location to ascertain where the user is when the rug is used. In addition, the system and app may comprise functionality chosen from Artificial intelligence (AI), Machine Learning, Metaverse, Web3, immersive technology, or combinations thereof.

The embodiments described herein are intended to be merely exemplary. Persons skilled in the art will understand that variations and modifications may be made without departing from the scope of the invention encompassed by the claims below.

I claim:

1. An integrated prayer rug system, comprising:
(a) a rug comprising an NFC chip in communication with an interface device having a computer processor and memory storing computer code wherein the interface device has internet communication capability and is chosen from a smartphone, a computer, a tablet computer, or a smartwatch; and
(b) an app to execute stored computer code on the interface device wherein the app is tailored to a specific user and comprises:
(i) a biometric authentication module to authenticate the user and to ensure digitally captured data is from the user;
(ii) a prayer and/or meditation module having a prayer and/or meditation plan and content for the user;
(iii) a user reminder module;
(iv) an application program interface (API) between the rug and the smartphone, the computer, the tablet computer, or the smartphone to link the rug to the app;
(v) a user self-assessment screen(s) module;
(vi) an interface device database module, comprising digitally-captured or user-entered prayer and/or meditation information, and information of the user's use of the rug and system;
(vii) a communications and reporting module to report user information on the smartphone, the computer, the tablet computer, or the smartphone, and to social media if the app is prompted to do so;
wherein the system utilizes information comprising prayer information, meditation information, the nature and extent of the user's use of the rug, user self-assessment information, digitally captured physiological, psychological, health and lifestyle information, information of prayers used by the user, or environmental data information;
wherein the system captures such information before, during or after each use of the system and which is utilized by the app to integrate with the user's overall health, exercise, and spiritual wellbeing.

2. The system according to claim 1, wherein information analyzed by the app are chosen from when the rug is used and for how long, goals, respiratory rate, blood oxygen saturation, pulse, heart rhythm, blood pressure, gait, muscle spasms, skin temperature, and weight.

3. The system according to claim 1, wherein data is captured by rug and/or the interface device and then prompts the user self-assessment module to ask the user one or more questions about his/her experience or condition.

4. The system according to claim 1, wherein the app stores the user's goals and/or schedule for prayer and/or meditation.

5. The system according to claim 1, wherein the app reminds the user to pray, meditate, and/or breathe.

6. The system according to claim 1, wherein the app is designed to automatically connect or handshake with the user's other digital health, exercise, lifestyle monitoring and/or tracking devices.

7. The system according to claim 1, wherein the app presents user specific self-assessment and/or data input screen(s).

8. The system according to claim 1, wherein the app maintains a database of user responses captured by self-assessment and/or data input screen(s) and digitally captured values and, optionally, scores the user's activity on the rug.

9. The system according to claim 1, wherein the app further comprises a GPS module to record the user's location to ascertain where the user is when the rug is used.

10. The system according to claim 1, wherein the app further comprises functionality chosen from Artificial intelligence (AI), Machine Learning, Metaverse, Web3, immersive technology, or combinations thereof.
